(19) United States
(12) Patent Application Publication
(10) Pub. No.: US 2005/0220726 A1
(43) Pub. Date: Oct. 6, 2005

(54) Use of sugar esters in cosmetic and/or pharmaceutical preparations

(76) Inventors: Gilles Pauly, Nancy (FR); Philippe Grisoni, Bey sur Seille (FR)

Correspondence Address: COGNIS CORPORATION;PATENT DEPARTMENT, 300 BROOKSIDE AVENUE, AMBLER, PA, 19002, US

(21) Appl. No.: 10/506,315

(22) PCT Filed: Feb. 20, 2003
(86) PCT No.: PCT/EP03/01731

§ 371 Date: Apr. 14, 2005

(51) Int. Cl. A61K 31/7024; A61K 7/42; A61K 7/06; A61K 7/11

(52) U.S. Cl. 424/59; 424/70.13; 514/23

(57) ABSTRACT

Methods for treating the skin and/or hair comprising: (a) providing a composition comprising a sugar ester; and (b) contacting a substrate selected from the group consisting of skin and hair with the composition are described.

### FIELD OF THE INVENTION

This invention relates generally to the field of cosmetics and, more particularly, to the use of sugar esters as active components for the production of hair and skin care preparations.

### PRIOR ART

The production of cosmetic preparations, particularly skin-care preparations, involves the use of active components distinguished by a complex requirement profile. They are expected to protect the skin in particular, but also the hair, against environmental poisons, oxidative stress and UV radiation, to prevent wrinkling, to show anti-inflammatory activity and, at the same time, to be particularly mild to the skin. Since the development and approval of such “cosmetic multipurpose weapons” is also very cost-intensive and time-consuming, there is a particular interest in active components which are already known and approved for use in cosmetic and pharmaceutical products or for which a production method has been described, but of which the potential as active-components has not hitherto been known or sufficiently researched.

By virtue of their particular dermatological compatibility, biodegradability and high emulsifiability, nonionic surfactants of the glycoside fatty acid ester type, more particularly fatty acid esters with sucrose, commonly referred to as sugar esters, have for many years enjoyed particular significance for numerous applications, such as for example the production of cosmetic emulsions, body care preparations, shampoos, hair sprays, toothpastes, lipsticks, mascaras and the like. The production and use of fatty acid esters of sucrose have been described particularly widely. The production and use of methyl glucose, fructose and trehalose have also been described. Their particular mildness, their contribution towards skin moisture regulation and their use for reducing the irritation potential of anionic surfactants or AHA are described, for example, by Desai in Cosm. Toil. 105, pp. 99-107 (1990) and in JP 960224502, EP 5904334 and JP 93168.

In this connection, reference is made to French patent application FR-A1 9211770 (L'Oréal) which discloses the use of fructose octanoate for restoring the lipid film on the skin and for reducing the transepidermal water loss (TEWL) of defatted skin. JP-A1 03/261711 and JP 03/197414 describe dextrose esters for improving the softness, combability and moisture of hair. JP-A1 09/241,404 (Lion) describes the use of esters of glucose with C₆₋₉ fatty acids against gram-positive bacteria and their use for the production of bactericidal preparations. EP-A1 0875239 and EP-A1 0985408 (BDF) proposes the use of esters of fatty acids with di- or oligosaccharides against the adhesion of microorganisms to hard surfaces.

International patent application WO 02/053121 describes cosmetic skin whitening preparations which contain glucose 3-5 acyl derivatives and/or sucrose 6-8 acyl derivatives containing acyl groups of 3 to 6 carbon atoms. Among these molecules, the skin whitening properties are particularly emphasized for glucose pentaisovalerate.

Accordingly, the problem addressed by the present invention was to identify glycoside fatty acid esters which, apart from their well-known properties, would be distinguished by a complex active-component profile. More particularly, these substances would protect the skin and hair against oxidative stress and environmental toxins. At the same time, they would be anti-inflammatory and would be particularly active against the germs involved in the development of Acne vulgaris.

### DESCRIPTION OF THE INVENTION

The present invention relates to the use of sugar esters as active components for the production of cosmetic and/or pharmaceutical preparations.

It has surprisingly been found that sugar esters, which have been known for many years as emulsifiers for foods, cosmetics and pharmaceutical preparations, have an extensive profile as an active component, even in very low concentrations. Accordingly, the present invention also relates to their use
hair growth inhibitors,
as active components for reducing the proliferation of keratinocytes,
as anti-inflammatory components,
as active components for protecting the skin and hair against the effect of UV radiation,
as active components for protecting the skin and hair against environmental toxins and oxidative stress,
as anti-ageing components,
as anti-protease components and more particularly as anti-collagenase components,
as active components for inhibiting the synthesis of melanin in skin and hair cells and hence as skin whiteners and
as anti-acne components.

It has been found that sugar esters (syn. glycoside fatty acid esters) are eminently suitable for inhibiting hair growth. They reduce the cell proliferation of human keratinoyctes and reduce the growth and the development of hair follicles.

The increasing trend in recent years to use depilatories, more particularly on the legs, in the subaxillary region or on the face, makes the use of preparations which reduce the rate of hair growth attractive, because even the intervals between the sometimes painful removal of hair can thus be lengthened.

Glycoside fatty acid esters improve the potential of the cells against oxidative stress and environmental toxins. Measurement of the cell-protecting (against oxidation or heavy metals, such as lead) glutathione (GSH) in human UV-A-damaged fibroblasts showed a distinct improvement in the GSH level after the treatment with monosaccharide or disaccharide fatty acid esters.

In addition, the sugar esters protect human keratinocytes against the harmful effects of UV-B radiation (measurable in a reduction in the release of LDH) and have an anti-inflammatory effect which is reflected in a reduction in the secretion of PGE2. In polymorphonuclear neutrophilic granulocytes, monosaccharide fatty acid esters show a significant inhibition of the respiratory burst effect—a release of reactive oxygen radicals involved in the inflammatory reaction. In addition, sugar esters inhibit protease activity, more particularly collagenase activity. It is known that collagenase activity increases with increasing age. Accordingly, sugar esters may therefore be used with advantage against ageing of the skin. Accordingly, they may also be used as against ageing of the cells by UV radiation, oxidative stress or environmental toxins which lead to increased collagenase activity.

In summary, therefore, sugar esters may be used with advantage for protecting skin and hair follicles against inflammation, sunburn, damage by radiation, oxidative stress, environmental toxins and ageing of the skin, particularly sensitive skin, and scalp.

It has also been found that sugar esters reduce the synthesis of melanin in B16 melanocytes, so that their use as skin whiteners is logical.

The presence of Propionibacterium acnes and Staphylococcus epidermidis lead to the known changes in the skin caused by acne. Propionibacterium acnes in particular causes increased comedone formation and promotes inflammatory reactions. It has now been found that not only are glycoside fatty acid esters effective against Staphylococcus epidermidis, they also suppress gram-negative bacteria, such as Propionibacterium acnes, and thus contribute significantly towards the anti-inflammatory effect in cases of acne.

Sugar Esters

Sugar esters are known nonionic surfactants which may be obtained by the relevant methods of preparative organic chemistry, for example by reaction of fatty acid methyl esters with corresponding sugars or enzymatically, as described for example in International patent application WO 99/02722 (Laboratoires Sérobiologiques). Sugar esters with different glycoside and acyl components are commercially obtainable, for example from the Sisterna and Ryoto companies. Typical examples of suitable sugar esters are shown below:

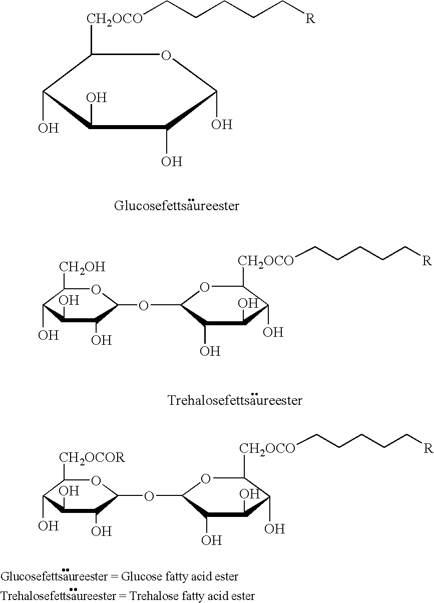

Basically, sugar esters derived from mono-, di- and/or oligosaccharides are suitable, including aldohexoses (for example glucose, methylglucose, mannose, galactose); deoxyaldoses (for example rhamnose, fucose, deoxyribose); aldopentoses (for example ribose, arabinose, xylose); ketoses (for example fructose in pyranosyl or furanosyl form); disaccharides (for example trehalose, sucrose, maltose, isomaltose, cellobiose, melibiose, gentobiose, lactose) and tri-, tetra-, penta- and oligosaccharides. Fructose, glucose, trehalose and/or sucrose esters are preferred, fructose esters being particularly preferred. The acyl component of the esters may be derived from fatty acids corresponding to formula (I):
R¹CO—OH (I)
in which R¹CO is a linear or branched, saturated or unsaturated acyl or hydroxyacyl group containing 6 to 22 and preferably 8 to 16 carbon atoms and 0 and/or 1 to 3 double bonds. Typical examples are sugar esters of caproic acid, 2-hyroxycaproic acid, 6-hydroxycaproic acid, caprylic acid, 2-ethylhexanoic acid, capric acid, 10-hydroxycaproic acid, lauric acid, 12-hydroxylauric acid, isotridecanoic acid, myristic acid, palmitic acid, palmitoleic acid, 16-hydroxypalmitic acid, stearic acid, isostearic acid, oleic acid, elaidic acid, petroselic acid, linoleic acid, linolenic acid, elaeostearic acid, 12-hydroxystearic acid, ricinoleic acid, arachic acid, gadoleic acid, behenic acid and erucic acid and technical mixtures thereof. The sugar-esters may also be derived from dicarboxylic acids containing 2 to 22 and preferably 6 to 18 carbon atoms, such as for example adipic acid or hexadecane dicarboxylic acid. The esters may contain 1 to 8 ester groups according to the hydroxyl groups available. However, products with an average degree of esterification of 1 to 6 and, more particularly, 1.5 to 2.5 are preferably used.
Cosmetic and/or Pharmaceutical Preparations

According to the invention, the sugar esters are used for the production of cosmertic and/or pharmaceutical preparations such as, for example, hair shampoos, hair lotions, foam baths, shower baths, creams, gels, lotions, alcoholic and aqueous/alcoholic solutions, emulsions, wax/fat compounds, stick preparations, powders or ointments, in which they may be present in quantities of 0.0001 to 10, preferably 0.001 to 5 and more particularly 0.01 to 1% by weight, based on the preparation. These preparations may also contain mild surfactants, oil components, emulsifiers, pearlizing waxes, consistency factors, thickeners, superfatting agents, stabilizers, polymers, silicone compounds, fats, waxes, lecithins, phospholipids, biogenic agents, UV protection factors, antioxidants, deodorants, antiperspirants, antidandruff agents, film formers, swelling agents, insect repellents, self-tanning agents, tyrosine inhibitors (depigmenting agents), hydrotropes, solubilizers, perservatives, perfume oils, dyes and the like as further auxiliaries and additives.

However, since sugar esters themselves have emulsifying, surface-active and moisturizing properties, they may also partly be used without additions of other surfactants or emulsifiers.

Surfactants

Suitable surfactants are anionic, nonionic, cationic and/or amphoteric or zwitterionic surfactants which may be present in the preparations in quantities of normally about 1 to 70% by weight, preferably 5 to 50% by weight and more preferably 10 to 30% by weight. Typical examples of anionic surfactants are soaps, alkyl benzenesulfonates, alkanesulfonates, olefin sulfonates, alkylether sulfonates, glycerol ether sulfonates, α-methyl ester sulfonates, sulfofatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids such as, for example, acyl lactylates, acyl tartrates, acyl glutamates and acyl aspartates, alkyl oligoglucoside sulfates, protein fatty acid condensates (particularly wheat-based vegetable products) and alkyl (ether) phosphates. If the anionic surfactants contain polyglycol ether chains, they may have a conventional homolog distribution although they preferably have a narrow-range homolog distribution. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partly oxidized alk(en)yl oligoglycosides or glucuronic acid derivatives, fatty acid-N-alkyl glucamides, protein hydrolyzates (particularly wheat-based vegetable products), polyol fatty acid esters, sugar esters, sorbitan esters, polysorbates and amine oxides. If the nonionic surfactants contain polyglycol ether chains, they may have a conventional homolog distribution, although they preferably have a narrow-range homolog distribution. Typical examples of cationic surfactants are quaternary ammonium compounds, for example dimethyl distearyl ammonium chloride, and esterquats, more particularly quaternized fatty acid trialkanolamine ester salts. Typical examples of amphoteric or zwitterionic surfactants are alkylbetaines, alkylamidobetaines, aminopropionates, aminoglycinates, imidazolinium betaines and sulfobetaines. The surfactants mentioned are all known compounds. Typical examples of particularly suitable mild, i.e. particularly dermatologically compatible, surfactants are fatty alcohol polyglycol ether sulfates, monoglyceride sulfates, mono- and/or dialkyl sulfosuccinates, fatty acid isethionates, fatty acid sarcosinates, fatty acid taurides, fatty acid glutamates, α-olefin sulfonates, ether carboxylic acids, alkyl oligoglucosides, fatty acid glucamides, alkylamidobetaines, amphoacetals and/or protein fatty acid condensates, preferably based on wheat proteins.

Oil Components

Suitable oil components are, for example, Guerbet alcohols based on fatty alcohols containing 6 to 18 and preferably 8 to 10 carbon atoms, esters of linear C₆₋₂₂ fatty acids with linear or branched C₆₋₂₂ fatty alcohols or esters of branched C₆₋₁₃ carboxylic acids with linear or branched C₆₋₂₂ fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆₋₂₂ fatty acids with branched alcohols, more particularly 2-ethyl hexanol, esters of C₁₈₋₃₈ alkylhydroxycarboxylic acids with linear or branched C₆₋₂₂ fatty alcohols, more especially Dioctyl Malate, esters of linear and/or branched fatty acids with polyhydric alcohols (for example propylene glycol, dimer diol or trimer triol) and/or Guerbet alcohols, triglycerides based on C₆₋₁₀ fatty acids, liquid mono-, di- and triglyceride mixtures based on C₆₋₁₈ fatty acids, esters of C₆₋₂₂ fatty alcohols and/or Guerbet alcohols with aromatic carboxylic acids, more particularly benzoic acid, esters of C₂₋₁₂ dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, linear and branched C₆₋₂₂ fatty alcohol carbonates, such as Dicaprylyl Carbonate (Cetiol® CC) for example, Guerbet carbonates based on C₆₋₁₈ and preferably C₈₋₁₀ fatty alcohols, esters of benzoic acid with linear and/or branched C₆₋₂₂ alcohols (for example Finsolv® TN), linear or branched, symmetrical or nonsymmetrical dialkyl ethers containing 6 to 22 carbon atoms per alkyl group, such as Dicaprylyl Ether (Cetiol® OE) for example, ring opening products of epoxidized fatty acid esters with polyols, silicone oils (cyclomethicone, silicon methicone types, etc.) and/or aliphatic or naphthenic hydrocarbons such as, for example, squalane, squalene or dialkyl cyclohexanes.

Emulsifiers

Suitable emulsifiers are, for example, nonionic surfactants from at least one of the following groups:
products of the addition of 2 to 30 mol ethylene oxide and/or 0 to 5 mol propylene oxide onto linear C₈₋₂₂ fatty alcohols, onto C₁₂₋₂₂ fatty acids, onto alkyl phenols containing 8 to 15 carbon atoms in the alkyl group and onto alkylamines containing 8 to 22 carbon atoms in the alkyl group;
alkyl and/or alkenyl oligoglycosides containing 8 to 22 carbon atoms in the alk(en)yl group and ethoxylated-analogs-thereof,
addition products of 1 to 15 mol ethylene oxide onto castor oil and/or hydrogenated castor oil;
addition products of 15 to 60 mol ethylene oxide onto castor oil and/or hydrogenated castor oil;
partial esters of glycerol and/or sorbitan with unsaturated, linear or saturated, branched fatty acids containing 12 to 22 carbon atoms and/or hydroxycarboxylic acids containing 3 to 18 carbon atoms and addition products thereof onto 1 to 30 mol ethylene oxide;
partial esters of polyglycerol (average degree of self-condensation 2 to 8), polyethylene glycol (molecular weight 400 to 5,000), trimethylolpropane, pentaerythritol, sugar alcohols (for example sorbitol), alkyl glucosides (for example methyl glucoside, butyl glucoside, lauryl glucoside) and polyglucosides (for example cellulose) with saturated and/or unsaturated, linear or branched fatty acids containing 12 to 22 carbon atoms and/or hydroxycarboxylic acids containing 3 to 18 carbon atoms and addition products thereof onto 1 to 30 mol ethylene oxide;
mixed esters of pentaerythritol, fatty acids, citric acid and fatty alcohol and/or mixed esters of fatty acids containing 6 to 22 carbon atoms, methyl glucose and polyols, preferably glycerol or polyglycerol,
mono-, di- and trialkyl phosphates and mono-, di- and/or tri-PEG-alkyl phosphates and salts thereof,
wool wax alcohols,
polysiloxane/polyalkyl/polyether copolymers and corresponding derivatives,
block copolymers, for example Polyethyleneglycol-30 Dipolyhydroxystearate;
polymer emulsifiers, for example Pemulen types (TR-1, TR-2) of Goodrich;
polyalkylene glycols and
glycerol carbonate.
   Ethylene Oxide Addition Products

The addition products of ethylene oxide and/or propylene oxide onto fatty alcohols, fatty acids, alkylphenols or onto castor oil are known commercially available products. They are homolog mixtures of which the average degree of alkoxylation corresponds to the ratio between the quantities of ethylene oxide and/or propylene oxide and substrate with which the addition reaction is carried out. C_{12/18} fatty acid monoesters and diesters of addition products of ethylene oxide onto glycerol are known as lipid layer enhancers for cosmetic formulations.

Alkyl and/or Alkenyl Oligoglycosides

Alkyl and/or alkenyl oligoglycosides, their production and their use are known from the prior art. They are produced in particular by reacting glucose or oligosaccharides with primary alcohols containing 8 to 18 carbon atoms. So far as the glycoside unit is concerned, both monoglycosides in which a cyclic sugar unit is attached to the fatty alcohol by a glycoside bond and oligomeric glycosides with a degree of oligomerization of preferably up to about 8 are suitable. The degree of oligomerization is a statistical mean value on which the homolog distribution typical of such technical products is based.

Partial Glycerides

Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride and technical mixtures thereof which may still contain small quantities of triglyceride from the production process. Addition products of 1 to 30 and preferably 5 to 10 mol ethylene oxide onto the partial glycerides mentioned are also suitable.

Sorbitan Esters

Suitable sorbitan esters are sorbitan monoisostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Addition products of 1 to 30 and preferably 5 to 10 mol ethylene oxide onto the sorbitan esters mentioned are also suitable.

Polyglycerol Esters

Typical examples of suitable polyglycerol esters are Polyglyceryl-2 Dipolyhydroxystearate (Dehymuls® PGPH), Polyglycerin-3-Diisostearate (Lameform® TGI), Polyglyceryl-4 Isostearate (Isolan® GI 34), Polyglyceryl-3 Oleate, Diisostearoyl Polyglyceryl-3 Diisostearate (Isolan® PDI), Polyglyceryl-3 Methylglucose Distearate (Tego Care® 450), Polyglyceryl-3 Beeswax (Cera Bellina®), Polyglyceryl-4 Caprate (Polyglycerol Caprate T2010/90), Polyglyceryl-3 Cetyl Ether (Chimexane® NL), Polyglyceryl-3 Distearate (Cremophor® GS 32) and Polyglyceryl Polyricinoleate (Admul® WOL 1403), Polyglyceryl Dimerate Isostearate and mixtures thereof. Examples of other suitable polyolesters are the mono-, di- and triesters of trimethylolpropane or pentaerythritol with lauric acid, cocofatty acid, tallow fatty acid, palmitic acid, stearic acid, oleic acid, behenic acid and the like optionally reacted with 1 to 30 mol ethylene oxide.

Anionic Emulsifiers

Typical anionic emulsifiers are aliphatic fatty acids containing 12 to 22 carbon atoms such as, for example, palmitic acid, stearic acid or behenic acid and dicarboxylic acids containing 12 to 22 carbon atoms such as, for example, azelaic acid or sebacic acid.

Amphoteric and Cationic Emulsifiers

Other suitable emulsifiers are zwitterionic surfactants. Zwitterionic surfactants are surface-active compounds which contain at least one quaternary ammonium group and at least one carboxylate and one sulfonate group in the molecule. Particularly suitable zwitterionic surfactants are the so-called betaines, such as the N-alkyl-N,N-dimethyl ammonium glycinates, for example cocoalkyl dimethyl ammonium glycinate, N-acylaminopropyl-N,N-dimethyl ammonium glycinates, for example cocoacylaminopropyl dimethyl ammonium glycinate, and 2-alkyl-3-carboxymethyl-3-hydroxyethyl imidazolines containing 8 to 18 carbon atoms in the alkyl or acyl group and cocoacylaminoethyl hydroxyethyl carboxymethyl glycinate. The fatty acid amide derivative known under the CTFA name of Cocamidopropyl Betaine is particularly preferred. Ampholytic surfactants are also suitable emulsifiers. Ampholytic surfactants are surface-active compounds which, in addition to a C_{8/18} alkyl or acyl group, contain at least one free amino group and at least one —COOH— or —SO₃H— group in the molecule and which are capable of forming inner salts. Examples of suitable ampholytic surfactants are N-alkyl glycines, N-alkyl propionic acids, N-alkylaminobutyric acids, N-alkyliminodipropionic acids, N-hydroxyethyl-N-alkylamidopropyl glycines, N-alkyl taurines, N-alkyl sarcosines, 2-alkylaminopropionic acids and alkylaminoacetic acids containing around 8 to 18 carbon atoms in the alkyl group. Particularly preferred ampholytic surfactants are N-cocoalkylaminopropionate, cocoacylaminoethyl aminopropionate and C_{12/18} acyl sarcosine. Finally, cationic surfactants are also suitable emulsifiers, those of the esterquat type, preferably methyl-quaternized difatty acid triethanolamine ester salts, being particularly preferred.

Fats and Waxes

Typical examples of fats are glycerides, i.e. solid or liquid, vegetable or animal products which consist essentially of mixed glycerol esters of higher fatty acids. Suitable waxes are inter alia natural waxes such as, for example, candelilla wax, carnauba wax, Japan wax, espartograss wax, cork wax, guaruma wax, rice oil wax, sugar cane wax, ouricury wax, montan wax, beeswax, shellac wax, spermaceti, lanolin (wool wax), uropygial fat, ceresine, ozocerite (earth wax), petrolatum, paraffin waxes and microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes. Besides the fats, other suitable additives are fat-like substances, such as lecithins and phospholipids. Lecithins are known among experts as glycerophospholipids which are formed from fatty acids, glycerol, phosphoric acid and choline by esterification. Accordingly, lecithins are also frequently referred to by experts as phosphatidyl cholines (PCs). Examples of natural lecithins are the kephalins which are also known as phosphatidic acids and which are derivatives of 1,2-diacyl-sn-glycerol-3-phosphoric acids. By contrast, phospholipids are generally understood to be mono- and preferably diesters of phosphoric acid with glycerol (glycerophosphates) which are normally classed as fats. Sphingosines and sphingolipids are also suitable.

Pearlizing Waxes

Suitable pearlizing waxes are, for example, alkylene glycol esters, especially ethylene glycol distearate; fatty acid alkanolamides, especially cocofatty acid diethanolamide; partial glycerides, especially stearic acid monoglyceride; esters of polybasic, optionally hydroxysubstituted carboxylic acids with fatty alcohols containing 6 to 22 carbon atoms, especially long-chain esters of tartaric acid; fatty compounds, such as for example fatty alcohols, fatty ketones, fatty aldehydes, fatty ethers and fatty carbonates which contain in all at least 24 carbon atoms, especially laurone and distearylether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups and mixtures thereof.

Consistency Factors and Thickeners

The consistency factors mainly used are fatty alcohols or hydroxyfatty alcohols containing 12 to 22 and preferably 16 to 18 carbon atoms and also partial glycerides, fatty acids or hydroxyfatty acids. A combination of these substances with alkyl oligoglucosides and/or fatty acid N-methyl glucamides of the same chain length and/or polyglycerol poly-12-hydroxystearates is preferably used. Suitable thickeners are, for example, Aerosil® types (hydrophilic silicas), polysaccharides, more especially xanthan gum, guar-guar, agar-agar, alginates and tyloses, carboxymethyl cellulose and hydroxyethyl cellulose, also relatively high molecular weight polyethylene glycol monoesters and diesters of fatty acids, polyacrylates (for example Carbopols® and Pemulen types [Goodrich]; Synthalens® [Sigma]; Keltrol types [Kelco]; Sepigel types [Seppic]; Salcare types [Allied Colloids]), polyacrylamides, polymers, polyvinyl alcohol and polyvinyl pyrrolidone. Other consistency factors which have proved to be particularly effective are bentonites, for example Bentone® Gel VS-5PC (Rheox) which is a mixture of cyclopentasiloxane, Disteardimonium Hectorite and propylene carbonate. Other suitable consistency factors are surfactants such as, for example, ethoxylated fatty acid glycerides, esters of fatty acids with polyols, for example pentaerythritol or trimethylol propane, narrow-range fatty alcohol ethoxylates or alkyl oligoglucosides and electrolytes, such as sodium chloride and ammonium chloride.

Superfatting Agents

Superfatting agents may be selected from such substances as, for example, lanolin and lecithin and also polyethoxylated or acylated lanolin and lecithin derivatives, polyol fatty acid esters, monoglycerides and fatty acid alkanolamides, the fatty acid alkanolamides also serving as foam stabilizers.

Stabilizers

Metal salts of fatty acids such as, for example, magnesium, aluminium and/or zinc stearate or ricinoleate may be used as stabilizers.

Polymers

Suitable cationic polymers are, for example, cationic cellulose derivatives such as, for example, the quaternized hydroxyethyl cellulose obtainable from Amerchol under the name of Polymer JR 400®, cationic starch, copolymers of diallyl ammonium salts and acrylamides, quaternized vinyl pyrrolidone/vinyl imidazole polymers such as, for example, Luviquat® (BASF), condensation products of polyglycols and amines, quaternized collagen polypeptides such as, for example, Lauryldimonium Hydroxypropyl Hydrolyzed Collagen (Lamequat® L, Grünau), quaternized wheat polypeptides, polyethyleneimine, cationic silicone polymers such as, for example, amodimethicone, copolymers of adipic acid and dimethylaminohydroxypropyl diethylenetriamine (Cartaretine®, Sandoz), copolymers of acrylic acid with dimethyl diallyl ammonium chloride (Merquat® 550, Chemviron), polyaminopolyamides and crosslinked water-soluble polymers thereof, cationic chitin derivatives such as, for example, quaternized chitosan, optionally in microcrystalline distribution, condensation products of dihaloalkyls, for example dibromobutane, with bis-dialkylamines, for example bis-dimethylamino-1,3-propane, cationic guar gum such as, for example, Jaguar®CBS, Jaguar®C-17, Jaguar®C-16 of Celanese, quaternized ammonium salt polymers such as, for example, Mirapol® A-15, Mirapol® AD-1, Mirapol® AZ-1 of Miranol.

Suitable anionic, zwitterionic, amphoteric and nonionic polymers are, for example, vinyl acetate/crotonic acid copolymers, vinyl pyrrolidone/vinyl acrylate copolymers, vinyl acetate/butyl maleate/isobornyl acrylate copolymers, methyl vinylether/maleic anhydride copolymers and esters thereof, uncrosslinked and polyol-crosslinked polyacrylic acids, acrylamidopropyl trimethylammonium chloride/acrylate copolymers, octylacrylamide/methyl methacrylate/tert.-butylaminoethyl methacrylate/2-hydroxypropyl methacrylate copolymers, polyvinyl pyrrolidone, vinyl pyrrolidone/vinyl acetate copolymers, vinyl pyrrolidone/dimethylaminoethyl methacrylate/vinyl caprolactam terpolymers and optionally derivatized cellulose ethers and silicones.

Silicone Compounds

Suitable silicone compounds are, for example, dimethyl polysiloxanes, methylphenyl polysiloxanes, cyclic silicones and amino-, fatty acid-, alcohol-, polyether-, epoxy-, fluorine-, glycoside- and/or alkyl-modified silicone compounds which may be both liquid and resin-like at room temperature. Other suitable silicone compounds are simethicones which are mixtures of dimethicones with an average chain length of 200 to 300 dimethylsiloxane units and hydrogenated silicates.

UV Protection Factors and Antioxidants

UV protection factors in the context of the invention are, for example, organic substances (light filters) which are liquid or crystalline at room temperature and which are capable of absorbing ultraviolet radiation and of releasing the energy absorbed in the form of longer-wave radiation, for example heat. UV-B filters can be oil-soluble or water-soluble. The following are examples of oil-soluble substances:
3-benzylidene camphor or 3-benzylidene norcamphor and derivatives thereof, for example 3-(4-methylbenzylidene)-camphor;
4-aminobenzoic acid derivatives, preferably 4-(dimethylamino)-benzoic acid-2-ethylhexyl ester, 4-(dimethylamino)-benzoic acid-2-octyl ester and 4-(dimethylamino)-benzoic acid amyl ester;
esters of cinnamic acid, preferably 4-methoxycinnamic acid-2-ethylhexyl ester, 4-methoxycinnamic acid propyl ester, 4-methoxycinnamic acid isoamyl ester, 2-cyano-3,3-phenylcinnamic acid-2-ethylhexyl ester (Octocrylene);
esters of salicylic acid, preferably salicylic acid-2-ethylhexyl ester, salicylic acid-4-isopropylbenzyl ester, salicylic acid homomenthyl ester;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably 4-methoxybenzalmalonic acid di-2-ethylhexyl ester;
triazine derivatives such as, for example, 2,4,6-trianilino-(p-carbo-2′-ethyl-1′-hexyloxy)-1,3,5-triazine and Octyl Triazone or Dioctyl Butamido Triazone (Uvasorb® HEB);
propane-1,3-diones such as, for example, 1-(4-tert.butylphenyl)-3-(4′-methoxyphenyl)-propane-1,3-dione;
ketotricyclo(5.2.1.0)decane derivatives.

Suitable water-soluble substances are
2-phenylbenzimidazole-5-sulfonic acid and alkali metal, alkaline earth metal, ammonium, alkylammonium, alkanolammonium and glucammonium salts thereof;
sulfonic acid derivatives of benzophenones, preferably 2-hydroxy-4-methoxybenzophenone-5-sulfonic acid and salts thereof;
sulfonic acid derivatives of 3-benzylidene camphor such as, for example, 4-(2-oxo-3-bornylidenemethyl)-benzene sulfonic acid and 2-methyl-5-(2-oxo-3-bornylidene)-sulfonic acid and salts thereof.

Typical UV-A filters are, in particular, derivatives of benzoyl methane such as, for example, 1-(4′-tert.butylphenyl)-3-(4′-methoxyphenyl)-propane-1,3-dione, 4-tert.butyl-4′-methoxydibenzoyl methane (Parsol® 1789) or 1-phenyl-3-(4′-isopropylphenyl)-propane-1,3-dione and enamine compounds. The UV-A and UV-B filters may of course also be used in the form of mixtures. Particularly favorable combinations consist of the derivatives of benzoyl methane, for example 4-tert.butyl-4′-methoxydibenzoylmethane (Parsol® 1789) and 2-cyano-3,3-phenylcinnamic acid-2-ethyl hexyl ester (Octocrylene) in combination with esters of cinnamic acid, preferably 4methoxycinnamic acid-2-ethyl hexyl ester and/or 4-methoxycinnamic acid propyl ester and/or 4-methoxycinnamic acid isoamyl ester. Combinations such as these are advantageously combined with water-soluble filters such as, for example, 2-phenylbenzimidazole-5-sulfonic acid and alkali metal, alkaline earth metal, ammonium, alkylammonium, alkanolammonium and glucammonium salts thereof.

Besides the soluble substances mentioned, insoluble light-blocking pigments, i.e. finely dispersed metal oxides or salts, may also be used for this purpose. Examples of suitable metal oxides are, in particular, zinc oxide and titanium dioxide and also oxides of iron, zirconium oxide, silicon, manganese, aluminium and cerium and mixtures thereof. Silicates (talcum), barium sulfate and zinc stearate may be used as salts. The oxides and salts are used in the form of the pigments for skin-care and skin-protecting emulsions and decorative cosmetics. The particles should have a mean diameter of less than 100 nm, preferably between 5 and 50 nm and more preferably between 15 and 30 nm. They may be spherical in shape although ellipsoidal particles or other non-spherical particles may also be used. The pigments may also be surface-treated, i.e. hydrophilicized or hydrophobicized. Typical examples are coated titanium dioxides, for example Titandioxid T 805 (Degussa) and Eusolex® T2000 (Merck). Suitable hydrophobic coating materials are, above all, silicones and, among these, especially trialkoxyoctylsilanes or simethicones. So-called micro- or nanopigments are preferably used in sun protection products. Micronized zinc oxide is preferably used.

Besides the two groups of primary sun protection factors mentioned above, secondary sun protection factors of the antioxidant type may also be used. Secondary sun protection factors of the antioxidant type interrupt the photochemical reaction chain which is initiated when UV rays penetrate into the skin. Typical examples are amino acids (for example glycine, histidine, tyrosine, tryptophane) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, α-carotene, lycopene) and derivatives thereof, chlorogenic acid and derivatives thereof, liponic acid and derivatives thereof (for example dihydroliponic acid), aurothioglucose, propylthiouracil and other thiols (for example thioredoxine, glutathione, cysteine, cystine, cystamine and glycosyl, N-acetyl, methyl, ethyl, propyl, amyl, butyl and lauryl, palmitoyl, oleyl, γ-linoleyl, cholesteryl and glyceryl esters thereof) and their salts, dilaurylthiodipropionate, distearylthiodipropionate, thiodipropionic acid and derivatives thereof (esters, ethers, peptides, lipids, nucleotides, nucleosides and salts) and sulfoximine compounds (for example butionine sulfoximines, homocysteine sulfoximine, butionine sulfones, penta-, hexa- and hepta-thionine sulfoximine) in very small compatible dosages (for example pmole to μmole/kg), also (metal) chelators (for example α-hydroxyfatty acids, palmitic acid, phytic acid, lactoferrine), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof (for example γ-linolenic acid, linoleic acid, oleic acid), folic acid and derivatives thereof, ubiquinone and ubiquinol and derivatives thereof, vitamin C and derivatives thereof (for example ascorbyl palmitate, Mg ascorbyl phosphate, ascorbyl acetate), tocopherols and derivatives (for example vitamin E acetate), vitamin A and derivatives (vitamin A palmitate) and coniferyl benzoate of benzoin resin, rutinic acid and derivatives thereof, α-glycosyl rutin, ferulic acid, furfurylidene glucitol, carnosine, butyl hydroxytoluene, butyl hydroxyanisole, nordihydroguaiac resin acid, nordihydroguaiaretic acid, trihydroxybutyrophenone, uric acid and derivatives thereof, mannose and derivatives thereof, Superoxid-Dismutase, zinc and derivatives thereof (for example ZnO, ZnSO₄), selenium and derivatives thereof (for example selenium methionine), stilbenes and derivatives thereof (for example stilbene oxide, trans-stilbene oxide) and derivatives of these active substances suitable for the purposes of the invention (salts, esters, ethers, sugars, nucleotides, nucleosides, peptides and lipids).

Biogenic Agents

In the context of the invention, biogenic agents are, for example, tocopherol, tocopherol acetate, tocopherol palmitate, ascorbic acid, (deoxy)ribonucleic acid and fragmentation products thereof, β-glucans, retinol, bisabolol, allantoin, phytantriol, panthenol, AHA acids, amino acids, ceramides, pseudoceramides, essential oils, plant extracts, for example prunus extract, bambara nut extract, and vitamin complexes.

Deodorants and Germ Inhibitors

Cosmetic deodorants counteract, mask or eliminate body odors. Body odors are formed through the action of skin bacteria on apocrine perspiration which results in the formation of unpleasant-smelling degradation products. Accordingly, deodorants contain active principles which act as germ inhibitors, enzyme inhibitors, odor absorbers or odor maskers.

Germ Inhibitors

Basically, suitable germ inhibitors are any substances which act against gram-positive bacteria such as, for example, 4-hydroxybenzoic acid and salts and esters thereof, N-(4-chlorophenyl)-N′-(3,4-dichlorophenyl)-urea, 2,4,4′-trichloro-2′-hydroxydiphenylether (triclosan), 4-chloro-3,5-dimethylphenol, 2,2′-methylene-bis-(6-bromo-4-chlorophenol), 3-methyl-4-(1-methylethyl)-phenol, 2-benzyl-4-chlorophenol, 3-(4-chlorophenoxy)-propane-1,2-diol, 3-iodo-2-propinyl butyl carbamate, chlorhexidine, 3,4,4′-trichlorocarbanilide (TTC), antibacterial perfumes, thymol, thyme oil, eugenol, clove oil, menthol, mint oil, farnesol, phenoxyethanol, glycerol monocaprate, glycerol monocaprylate, glycerol monolaurate (GML), diglycerol monocaprate (DMC), salicylic acid-N-alkylamides such as, for example, salicylic acid-n-octyl amide or salicylic acid-n-decyl amide.

Enzyme Inhibitors

Suitable enzyme inhibitors are, for example, esterase inhibitors. Esterase inhibitors are preferably trialkyl citrates, such as trimethyl citrate, tripropyl citrate, triisopropyl citrate, tributyl citrate and, in particular, triethyl citrate (Hydagen® CAT). Esterase inhibitors inhibit enzyme activity and thus reduce odor formation. Other esterase inhibitors are sterol sulfates or phosphates such as, for example, lanosterol, cholesterol, campesterol, stigmasterol and sitosterol sulfate or phosphate, dicarboxylic acids and esters thereof, for example glutaric acid, glutaric acid monoethyl ester, glutaric acid diethyl ester, adipic acid, adipic acid monoethyl ester, adipic acid diethyl ester, malonic acid and malonic acid diethyl ester, hydroxycarboxylic acids and esters thereof, for example citric acid, malic acid, tartaric acid or tartaric acid diethyl ester, and zinc glycinate.

Odor Absorbers

Suitable odor absorbers are substances which are capable of absorbing and largely retaining the odor-forming compounds. They reduce the partial pressure of the individual components and thus also reduce the rate at which they spread. An important requirement in this regard is that perfumes must remain unimpaired. Odor absorbers are not active against bacteria. They contain, for example, a complex zinc salt of ricinoleic acid or special perfumes of largely neutral odor known to the expert as “fixateurs” such as, for example, extracts of ladanum or styrax or certain abietic acid derivatives as their principal component. Odor maskers are perfumes or perfume oils which, besides their odor-masking function, impart their particular perfume note to the deodorants. Suitable perfume oils are, for example, mixtures of natural and synthetic fragrances. Natural fragrances include the extracts of blossoms, stems and leaves, fruits, fruit peel, roots, woods, herbs and grasses, needles and branches, resins and balsams. Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, p-tert.butyl cyclohexylacetate, linalyl acetate, phenyl ethyl acetate, linalyl benzoate, benzyl formate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable fragrance. Other suitable perfume oils are essential oils of relatively low volatility which are mostly used as aroma components. Examples are sage oil, camomile oil, clove oil, lemon balm oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Antiperspirants

Antiperspirants reduce perspiration and thus counteract underarm wetness and body odor by influencing the activity of the eccrine sweat glands. Aqueous or water-free antiperspirant formulations typically contain the following ingredients:
astringent active principles,
oil components,
nonionic emulsifiers,
co-emulsifiers,
consistency factors,
auxiliaries in the form of, for example, thickeners or complexing agents and/or
non-aqueous solvents such as, for example, ethanol, propylene glycol and/or glycerol.

Suitable astringent active principles of antiperspirants are, above all, salts of aluminium, zirconium or zinc. Suitable antihydrotic agents of this type are, for example, aluminium chloride, aluminium chlorohydrate, aluminium dichlorohydrate, aluminium sesquichlorohydrate and complex compounds thereof, for example with 1,2-propylene glycol, aluminium hydroxyallantoinate, aluminium chloride tartrate, aluminium zirconium trichlorohydrate, aluminium zirconium tetrachlorohydrate, aluminium zirconium pentachlorohydrate and complex compounds thereof, for example with amino acids, such as glycine. Oil-soluble and water-soluble auxiliaries typically encountered in antiperspirants may also be present in relatively small amounts. Oil-soluble auxiliaries such as these include, for example,
inflammation-inhibiting, skin-protecting or pleasant-smelling essential oils,
synthetic skin-protecting agents and/or
oil-soluble perfume oils.

Typical water-soluble additives are, for example, preservatives, water-soluble perfumes, pH adjusters, for example buffer mixtures, water-soluble thickeners, for example water-soluble natural or synthetic polymers such as, for example, xanthan gum, hydroxyethyl cellulose, polyvinyl pyrrolidone or high molecular weight polyethylene oxides.

Film Formers

Standard film formers are, for example, chitosan, microcrystalline chitosan, quaternized chitosan, polyvinyl pyrrolidone, vinyl pyrrolidone/vinyl acetate copolymers, polymers of the acrylic acid series, quaternary cellulose derivatives, collagen, hyaluronic acid and salts thereof and similar compounds.

Antidandruff Agents

Suitable antidandruff agents are Pirocton Olamin (1-hydroxy-4-methyl-6-(2,4,4-trimethylpentyl)-2-(1H)-pyridinone monoethanolamine salt), Baypival® (Climbazole), Ketoconazol® (4-acetyl-1-{4-[2-(2,4-dichlorophenyl) r-2-(1H-imidazol-1-ylmethyl)-1,3-dioxylan-c-4-ylmethoxyphenyl}-piperazine, ketoconazole, elubiol, selenium disulfide, colloidal sulfur, sulfur polyethylene glycol sorbitan monooleate, sulfur ricinol polyethoxylate, sulfur tar distillate, salicylic acid (or in combination with hexachlorophene), undecylenic acid, monoethanolamide sulfosuccinate Na salt, Lamepon® UD (protein/undecylenic acid condensate), zinc pyrithione, aluminum pyrithione and magnesium pyrithione/dipyrithione magnesium sulfate.

Swelling Agents

Suitable swelling agents for aqueous phases are montmorillonites, clay minerals, Pemulen and alkyl-modified Carbopol types (Goodrich).

Insect Repellents

Suitable insect repellents are N,N-diethyl-m-toluamide, pentane-1,2-diol or Ethyl Butylacetylaminopropionate.

Self-Tanning Agents and Depigmenting Agents

A suitable self-tanning agent is dihydroxyacetone. Suitable tyrosine inhibitors which prevent the formation of melanin and are used in depigmenting agents are, for example, arbutin, ferulic acid, koji acid, coumaric acid and ascorbic acid (vitamin C).

Hydrotropes

In addition, hydrotropes, for example ethanol, isopropyl alcohol or polyols, may be used to improve flow behavior. Suitable polyols preferably contain 2 to 15 carbon atoms and at least two hydroxyl groups. The polyols may contain other functional groups, more especially amino groups, or may be modified with nitrogen. Typical examples are
glycerol;
alkylene glycols such as, for example, ethylene glycol, diethylene glycol, propylene glycol, butylene glycol, hexylene glycol and polyethylene glycols with an average molecular weight of 100 to 1000 dalton;
technical oligoglycerol mixtures with a degree of self-condensation of 1.5 to 10 such as, for example, technical diglycerol mixtures with a diglycerol content of 40 to 50% by weight;
methylol compounds such as, in particular, trimethylol ethane, trimethylol propane, trimethylol butane, pentaerythritol and dipentaerythritol;
lower alkyl glucosides, particularly those containing 1 to 8 carbon atoms in the alkyl group, for example methyl and butyl glucoside;
sugar alcohols containing 5 to 12 carbon atoms, for example sorbitol or mannitol,
sugars containing 5 to 12 carbon atoms, for example glucose or sucrose;
amino sugars, for example glucamine;
dialcoholamines, such as diethanolamine or 2-aminopropane-1,3-diol.
   Preservatives

Suitable preservatives are, for example, phenoxyethanol, formaldehyde solution, parabens, pentanediol or sorbic acid and the silver complexes known under the name of Surfacine® and the other classes of compounds listed in Appendix 6, Parts A and B of the Kosmetikverordnung (“Cosmetics Directive”).

Perfume Oils and Aromas

Suitable perfume oils are mixtures of natural and synthetic perfumes. Natural perfumes include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamom, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert.butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyloxyacetaldehyde, cyclamen aldehyde, hydroxycitronellal, lilial and bourgeonal. Examples of suitable ketones are the ionones, α-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable perfume. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Suitable aromas are, for example, peppermint oil, spearmint oil, aniseed oil, Japanese anise oil, caraway oil, eucalyptus oil, fennel oil, citrus oil, wintergreen oil, clove oil, menthol and the like.

Dyes

Suitable dyes are any of the substances suitable and approved for cosmetic purposes as listed, for example, in the publication “Kosmetische Färbemittel” of the Farbstoffkommission der Deutschen Forschungs-gemeinschaft, Verlag Chemie, Weinheim, 1984, pages 81 to 106. Examples include cochineal red A (C.I. 16255), patent blue V (C.I. 42051), indigotin (C.I. 73015), chlorophyllin (C.I. 75810), quinoline yellow (C.I. 47005), titanium dioxide (C.I. 77891), indanthrene blue RS(C.I. 69800) and madder lake (C.I. 58000). Luminol may also be present as a luminescent dye. These dyes are normally used in concentrations of 0.001 to 0.1% by weight, based on the mixture as a whole.

The total percentage content of auxiliaries and additives may be from 1 to 50% by weight and is preferably from 5 to 40% by weight, based on the particular preparations. The preparations may be produced by standard hot or cold processes and are preferably produced by the phase inversion temperature method.

### EXAMPLES

Enzymatically prepared sugar esters of fructose, glucose and trehalose which had been prepared in accordance with International patent application WO 99/02722 (Laboratoires Sérobiologiqes) were used in the following Examples. They were purified by liquid/liquid extraction or extraction with supercritical carbon dioxide. The sucrose esters (SE) used were commercial products of the Sisterna and Ryoto companies. The exact composition of the esters is shown in Table 1:

TABLE 1
Composition of the sugar esters used

| | | | Residual fatty |
|---|---|---|---|
| | Mono/diester | Residual sugar | acid content, |
| Sugar ester | ratio | content, % by wt. | % by wt. |
| Fructose caprate | 58:42 | 5 | <5 |
| Fructose dicaprate | 9:91 | <3 | <5 |
| Fructose palmitate | 49:51 | <3 | <5 |
| Fructose stearate | 49:51 | <3 | <3 |
| Fructose monostearate | >98:<2 | <3 | <3 |
| Glucose caprylate | >98:<2 | <5 | 11 |
| Glucose laurate | >98:<2 | <5 | 8 |
| Glucose palmitate | >98:<2 | <5 | <5 |
| Trehalose caprate | 57:43 | <5 | <5 |
| Trehalose laurate | 33:77 | <5 | <5 |
| Trehalose palmitate | 52:48 | <5 | <5 |
| Trehalose stearate | 48:52 | <5 | <5 |

A) Regenerative Effect and Detoxifying Potential

Glutathione (GSH) is a special protein which is produced by the cells for protection against oxidative stress and environmental poisons, more particularly against heavy metals. The three amino acids involved in the reduced form of GSH are linked to special cytoplasmatic enzymes which need ATP for activation. An increase in the GSH concentration leads to an increase in the glutatione-S-transferase activity, a detoxifying enzyme. The stimulation for detoxification by the test substances was tested on human fibroblasts by measurement of the GSH. In a series of tests, fibroblasts were first incubated for 3 days at 37° C. in a nutrient solution and then for 3 days at the same temperature in a test solution. The protein content in the cells was then determined by the Bradford method and the GSH concentration by the Hissin method [cf. Analytical Biochem. 74, 214-226 1977]. The results are set out in Table 2, where they are expressed in %-rel. against a blank sample, and represent the results of 3 series of measurements with triple determination.

TABLE 2
Growth-and survival-stimulating effect (figures = %-rel.)

| Sugar ester | Conc.. % w/v | GSH/proteins |
|---|---|---|
| Blank sample | 0 | 100 |
| Trehalose palmitate | 0.001 | 125 |
| Trehalose stearate | 0.001 | 141 |

The Examples show that the test substances stimulate the metabolism in regard to growth and protection in the form of the detoxifying potential of the fibroblasts.

B) Anti-Stress Effect on UV-A-Damaged Fibroblasts

UV-A radiation in the 320 to 400 nm range induces oxidative stress which is mainly produced by the activation of photosensitive biological components which in turn catalyze the formation of ROS-like superoxide anions, hydrogen peroxide and singlet oxygen atoms. The anti-UV-A effect was tested on fibroblasts because UV-A radiation penetrates through the dermis where it causes oxidative damage by lipoperoxidation of the cell membrane and a reduction in the content of reduced glutathione (GHS). To this end, human fibroblasts were cultivated as described in a), exposed to UV-A rays (20 J/cm²) and the cell counts and the GSH content subsequently determined. The results are set out in Table 3, where they are expressed in %-rel. against a blank sample, and represent the results of 3 series of measurements with triple determination.

TABLE 3
Anti-stress effect (figures = %-rel.)

| | Conc. | | |
|---|---|---|---|
| Sugar ester | % w/v | Cell proteins | GSH/proteins |
| Blank sample | 0 | 100 | 100 |
| Blank sample + UV-A | 0 | 105 | 65 |
| Fructose palmitate + UV-A | 0.003 | 91 | 81 |
| Fructose stearate + UV-A | 0.003 | 83 | 79 |
| Glucose caprate + UV-A | 0.003 | 109 | 144 |
| Glucose laurate + UV-A | 0.0001 | 95 | 91 |
| Glucose palmitate + UV-A | 0.003 | 119 | 114 |
| Trehalose caprate + UV-A | 0.00003 | 120 | 87 |
| Trehalose laurate + UV-A | 0.003 | 119 | 90 |
| Trehalose palmitate + UV-A | 0.001 | 124 | 87 |
| Trehalose stearate + UV-A | 0.001 | 124 | 84 |

The GSH content of the fibroblasts was protected against UV-A radiation by the use of the sugar esters.

C) Protecting Cells Against UVB Radiation

The function of this test was to show that the test substances have anti-inflammatory properties for human keratinocytes. UVB was selected as the stress factor because the rays produce cutaneous inflammation (erythemas, oedemas) by activating enzymes that release arachidonic acid, such as phospholipase A2 (PLA2) for example. This results not only in damage to the membranes, but also in the formation of inflammatory substances, such as prostaglandins of the PGE2 type for example. The influence of UVB rays on keratinocytes was determined in vitro through the release of cytoplasmatic enzymes, such as LDH (lactate dehydrogenase) for example, which runs parallel to the cell damage and the formation of PGE2. To carry out the test, a fibroblast culture was mixed with foetal calf serum and inoculated with the test substances 2 days later. After incubation for 36 h at 37° C. and a CO₂ level of 5% by vol., the nutrient medium was replaced by an electrolyte solution and the fibroblasts were damaged with a particular dose of UVB (50 mJ/cm²). The quantity of keratinocytes was determined after trypsination via a cell counter while the LDH concentration was enzymatically determined. The results are set out in Table 4, where they are expressed as the activity in %-rel. against a standard, and represent the mean value of two test series involving double determination.

TABLE 4
Effect against UVB rays (figures = %-rel.)

| | Conc. | | LDH | PGE2 |
|---|---|---|---|---|
| Sugar ester | % w/v | Cell DNA | released | released |
| Blank sample | 0 | 100 | 0 | 0 |
| Control + UV-B | 0 | 33 | 100 | 100 |
| Fructose caprate + UV-B | 0.001 | 32 | 68 | 35 |
| | 0.003 | 140 | 0 | 0 |
| Glucose caprate + UV-B | 0.003 | 28 | 72 | 50 |
| | 0.01 | 36 | 48 | 28 |
| Glucose laurate + UV-B | 0.001 | 17 | 105 | 107 |
| | 0.003 | 54 | 49 | 23 |
| Glucose palmitate + UV-B | 0.001 | 32 | 68 | 63 |
| | 0.003 | 32 | 68 | 65 |

The results show that the test substances significantly reduce the harmful effects of UVB rays and, in particular, reduce the release of LDH and PGE2.

D) Anti-Inflammatory Activity

In the course of cutaneous inflammation, leucocytes, such as the polymorphonuclear neutrophilic granulocytes (PMNs) for example, are stimulated by peptides, such as cytokines for example, to emit messenger substances, such as leucotriene for example, which are released from activated or necrotic cells in the dermis. These activated PMNs release not only pro-inflammatory cytokinins, leucotrienes and proteases, but also ROS, such as superoxides and hypochlorite anions for example, of which the function is to destroy penetrated pathogenic germs or fungi. This activity of the PMNs during the inflammation is known as so-called respiratory burst. To investigate to what extent the test extracts can prevent or reduce the respiratory burst, a cell line of human leukaemic granulocytes of these PMNs was incubated together with the test substances at 37° C. and 5% by vol. CO₂. After the respiratory burst had been initiated by addition of a yeast extract (zymosan) to the cell solution, the release of superoxide anions was determined through their reaction with luminol. The results are set out in Table 5 which shows the cell counts and the quantity of ROS released in %-rel to the standard as the mean value of a series of measurements involving triple determination.

TABLE 5
Anti-inflammatory activity (figures = %-rel.)

| | Conc. | | |
|---|---|---|---|
| Sugar ester | % w/v | Cell counts | ROS released |
| Blank sample + stimulation | 0 | 100 | 100 |
| Fructose caprate + stimulation | 0.003 | 97 | 87 |
| | 0.01 | 60 | 5 |
| Fructose palmitate + stimulation | 0.001 | 97 | 81 |
| | 0.01 | 98 | 42 |
| Fructose stearate + stimulation | 0.001 | 100 | 88 |
| | 0.01 | 155 | 26 |
| Glucose palmitate + stimulation | 0.001 | 102 | 99 |
| | 0.01 | 91 | 58 |

The results show that the test substances have a strong inhibiting influence on the respiratory burst of human granulocytes but do not damage the granulocytes.

D) Effectiveness Against Proteases

In the event of inflammation, skin proteases, for example collagenase, are released from the polymorphonuclear neutrophilic granulocytes or macrophages. A similar process takes place in the skin of elderly people on exposure to UV rays. As already mentioned, the proteases—also known as matrix metalloproteases (MMPs) through their content of central zinc ions—catalyze the fragmentation of connective tissue proteins. The test substances were tested for collagenase inhibition using bacterial collagenase (clostridium histolyticum) on gelatin marked with fluorochromium (FITC, Calbiochem) as a natural nutrient medium. The incubation time was 60 mins. at 20° C.; the hydrolysis of the substrate was monitored by fluorescence at 393 nm (excitation at 328 nm). The results are set out in Table 6 as the collagenase inhibition in %.

TABLE 6
Collagenase inhibition (figures = %-rel.)

| | | Collagenase |
|---|---|---|
| Sugar ester | Conc. [w/v] | inhibition |
| Blank sample | 0 | 0 |
| Fructose palmitate | 0.03 | 4 |
| | 0.1 | 41 |
| | 0.3 | 78 |
| Fructose stearate | 0.3 | 45 |
| Glucose laurate | 0.1 | 28 |
| Trehalose caprate | 0.3 | 52 |
| Trehalose laurate | 0.1 | 21 |
| Trehalose palmitate | 0.3 | 9 |

The results show that the test substances have a significant inhibiting effect depending on concentration. To this extent, the glycoside fatty acid esters tested can be successfully used again ageing of the skin because there is a distinct increase in collagenase activity during the ageing process. In addition, they can be used against ageing of the skin accelerated in particular by oxidative stress, UV radiation or environmental poisons because it is known that these factors in particular promote the release of active collagenase.

F) Inhibition of Melanin Synthesis in B16 Melanocytes

Melanin is the pigment responsible for the color of the skin and hair. It is formed in special organelles, the melanosomes, which occur in the melanocytes of the basal layer of the human epidermis. The biosynthesis of melanin begins with the amino acid tyrosine which is oxidized to DOPA (dihydroxyphenylalanine) in the presence of tyrosinase. The DOPA then polymerizes to melanin. In order to demonstrate the inhibition of melanin synthesis, melanocytes (B16 cell line) inoculated in a standard medium. After incubation for 3 days at 37° C./5% CO₂, the nutrient medium was replaced by a solution containing the test substances in various concentrations. After incubation for another 3 days, the cell protein content (Bradford's method) and the content of synthesized melanin were determined through the optical density of the homogenizate at 475 nm. The results are expressed in % against a blank value and are set out in Table 7.

TABLE 7
Melanin synthesis in B16 melanocytes [figures = %-rel.]

| | Conc. | Cell | Cell |
|---|---|---|---|
| Sugar ester | % w/v | proteins | melanin |
| Blank value | 0 | 100 | 100 |
| Fructose caprate | 0.003 | 108 | 54 |
| | 0.006 | 80 | 30 |
| Fructose dicaprate | 0.003 | 105 | 88 |
| | 0.01 | 91 | 41 |
| Fructose stearate | 0.006 | 105 | 49 |
| | 0.01 | 76 | 21 |
| Fructose monostearate | 0.001 | 104 | 85 |
| | 0.003 | 91 | 34 |
| Trehalose caprate | 0.001 | 100 | 53 |
| | 0.003 | 75 | 23 |
| Trehalose stearate | 0.001 | 97 | 81 |
| | 0.003 | 93 | 41 |
| Sucrose stearate¹⁾ | 0.001 | 89 | 75 |
| | 0.003 | 70 | 18 |

¹⁾Ryoto S1670

The results show that the test substances significantly inhibit the synthesis of melanin in the B16 melanocytes.

G) Growth Inhibition on Human Keratinocytes

Increased proliferation and differentiation of keratinocytes of the hair matrix leads to improved longer hair growth. The following test was carried out to determine the potential of the sugar esters for inhibiting hair growth which is visibly reflected in a reduction in the proliferation of human keratinocyte culture in vitro.

To this end, human keratinocytes were cultivated in a standard cell culture medium containing foetal calf serum (FCS). After incubation for 1 day at 37° C./5% CO₂, the growth medium was replaced by standard medium containing 10 ng/ml epidermal growth factor (EGF) and various concentrations of sugar esters dissolved in ethanol (1% by volume).

After incubation for three days, the number of living cells was determined by determining the cell protein content by the Bradford method [Bradford, M. M., A rapid and sensitive method for the quantitation of microgram quantities of protein utilizing the principle of protein-dye binding. Anal. Biochem. (1977), Vol. 72, pp. 248-254].

The results of Table 8 represent the values of triple determinations on two batches expressed in % against a control of cell culture medium without sugar ester.

TABLE 8
Growth inhibition on human keratinocytes (in % versus control)

| | | Cell protein |
|---|---|---|
| Sugar ester | Conc. % w/v | content in % |
| Control* | 0 | 100 |
| Control with EGF* | 0 | 167 |
| Fructose caprate with EGF | 0.001 | 163 |
| | 0.003 | 130 |
| | 0.01 | 45 |
| Fructose palmitate with EGF | 0.001 | 166 |
| | 0.003 | 143 |
| | 0.01 | 105 |

*Control experiments were carried out using 1% by vol. ethanol without sugar ester. They also showed that ethanol did not have a significant effect on the growth behavior of the keratinocytes.

The sugar esters tested significantly reduced the cell proliferation of human keratinocytes cultivated in vitro with EGF. Accordingly, they demonstrated a high potential for inhibiting hair growth.

H) Inhibition of Hair Growth

The hair-growth-inhibiting effect of the sugar esters was determined by a method using in vitro cultivated human hair follicles in a growth medium. The growth of the hair follicles was tested in the presence and absence of fructose caprate. To this end, the hair follicles were incubated for 7 days at 37° C./5% CO₂. The length of the cultivated hair follicles was recorded after 3 days and 7 days of cultivation. In a comparison example, the growth medium additionally contained 30 ng/ml IGF-1 insulin-like growth factor), a cytokine which stimulates hair follicle growth.

Each of the tests was carried out with 10 hair follicles of a donor (5 donors in all).

The results are set out in Table 9 and represent the mean values of the 50 hair follicles.

TABLE 9
Hair follicle growth - increase in hair growth after 3 and 7 days'
incubation - based on the starting length (day 0)

| Follicle treatment | Conc. % w/v | Day 3/day 0 | Day 7/day 0 |
|---|---|---|---|
| Control | 0 | +15% | +32% |
| Fructose caprate | 0.01 | +12% | +21% |
| Control | 0 | +21% | +45% |
| Fructose caprate + IGF | 0.01 | +15% | +25% |

The results show a distinct reduction in hair growth in the absence of IGF after treatment with the sugar esters tested in a concentration of only 0.01% by weight. The difference is even clearer where the IGF-containing solution is used.

I) Antibacterial Activity

The antibacterial activity of the test substances was tested by the diffusion method on agar plates or the agar dilution method. In this method, a round filter paper of defined diameter is first impregnated with the test solution and then applied to the surface of an agar plate inoculated beforehand with the test microorganisms. The size of the inhibition zones is then determined after defined times. In particular, it is possible by this method to determine the MIC (Minimum Inhibitory Concentration) as the lowest concentration of test substance with which complete inhibition of the microorganisms can be obtained.

Agar diffusion method. The inoculum was prepared using a fresh culture in a stationary growth phase (after ca. 18-24 h) in a BHI solution (brain-heart Infusion). The bacterial suspension was adjusted to 0.5 MacFarland units, corresponding to 1.5 10⁸ colony-forming units (cfu)/ml; the suspension was then diluted with a sodium chloride solution (1:100) to adjust a value of 1.5 10⁶ cfu/ml. Mueller-Hinton agar (Staphylococcus epidermidis, Staphylococcus aureus) and Wilkins-Chalgrens agar containing 5% by weight sheep's blood (Propionibacterium acnes) were then sterilized for 15 mins. at 121° C. and then placed in Petri dishes. 2 to 4 ml of the bacterial suspension were then added to the Petri dishes, followed by drying at room temperature. The filter papers were impregnated with 20 μl of the test substances and applied to the surface of the agar plates. The inoculated Petri dishes were incubated for 18 to 24 h at 37° C. (the Petri dishes containing Propionibacterium acnes under anaerobic conditions) and the antimicrobial activity was then determined by determination of the growth inhibition zones. The results are set out in Table 10 and represent the diameters of the inhibition zones in mm.

Agar dilution method (MIC determination). Various agars were prepared as described above, test substances were added in various concentrations, homogenized and dried. The Petri dishes were then inoculated with quantities of 2 μl of the bacterial suspension. After each drying step the Petri dishes were incubated for 18 to 24 h at 37° C. Table 11 shows the MIC in mg/ml, i.e. the lowest concentrations with which complete inhibition of the bacterial growth can be achieved. The results represent the averages of double determinations.

TABLE 10
Inhibition zones [mm]

| | Staphylococcus | Staphylococcus | Propionibacterium |
|---|---|---|---|
| Sugar ester | aureus | epidermidis | acnes |
| Fructose caprate | 10 | 9 | 14 |
| Glucose caprylate | 11 | 0 | 12 |
| Glucose laurate | 14 | 16 | 10 |
| Trehalose caprate | 10 | 0 | 10 |
| Trehalose | 14 | 11 | 0 |
| palmitate | | | |
| Trehalose stearate | 12 | 11 | 0 |

TABLE 11
MIC [mg/ml]

| | Staphylococcus | Staphylococcus | Propionibacterium |
|---|---|---|---|
| Sugar ester | epidermidis | aureus | acnes |
| Fructose caprate | 1.25 | 1.25 | 0.625 |
| Trehalose stearate | 0.625 | >10 | 1.25 |

The results show that the test substances have excellent antimicrobial properties, particularly against those germs which are involved in the development of acne.

A number of Formulation Examples are set out in the following Table.

TABLE 12
White emulsions containing sugar esters as emulsifiers
(figures = % by weight)

| Phase | Ingredient | A1 | A2 | A3 | A4 |
|---|---|---|---|---|---|
| Phase I | Cetearyl Alcohol | 3 | 3 | 3 | 3 |
| | Cetearyl Isononanoate | 15 | 15 | 15 | 15 |
| | Caprylic Capric | 6 | 6 | 6 | 6 |
| | Triglycerides | | | | |
| | Fructose palmitate | 3 | — | — | — |
| | Trehalose palmitate | — | 3 | — | — |
| | Trehalose stearate | — | — | 3 | — |
| | Sucrose stearate¹⁾ | — | — | — | 3 |
| Phase 2 | Water | to 100 | | | |
| | Elestab ® 388 | 2.5 | 2.5 | 2.5 | 2.5 |
| | Keltrol ® T | 0.2 | 0.2 | 0.2 | 0.2 |
| | NaOH 1 N | 0.2 | 0.2 | 0.2 | — |
| Phase 3 | Citric acid (10% by wt.) | — | 0.1 | 0.1 | — |

¹⁾Sisterna SP50C

To prepare the emulsion, phase 1 was introduced first at 75° C. and phase 2—heated to the same temperature—was added with vigorous stirring. After cooling to room temperature, phase 3 was stirred in. After dilution to 5% by weight, the emulsions had a pH of 6.2 to 7.8 and a Brookfield viscosity of 120 to 600 μs.

TABLE 13
White emulsions containing sugar esters as co-emulsifiers
(figures = % by weight)

| Phase | Ingredient | B1 | B2 | B3 | B4 | B5 | B6 |
|---|---|---|---|---|---|---|---|
| Phase 1 | Cetearyl Alcohol | 5 | 5 | 5 | 5 | 5 | 5 |
| | (and) Ceteareth 20 | | | | | | |
| | Cetearyl Alcohol | 3 | 3 | 3 | 3 | 3 | 3 |
| | Cetearyl | 15 | 15 | 15 | 15 | 15 | 15 |
| | Isononanoate | | | | | | |
| | Caprylic Capric | 3 | 3 | 3 | 3 | 3 | 3 |
| | Triglycerides | | | | | | |
| | Fructose caprate | 3 | — | — | — | — | — |
| | Glucose caprylate | — | 3 | — | — | — | — |
| | Glucose laurate | — | — | 3 | — | — | — |
| | Trehalose caprate | — | — | — | 3 | — | — |
| | Trehalose laurate | — | — | — | — | 3 | — |
| | Sucrose laurate¹⁾ | — | — | — | — | — | 3 |
| Phase 2 | Water | to 100 | | | | | |
| | Elestab ® 388 | 2.5 | 2.5 | 2.5 | 2.5 | 2.5 | 2.5 |
| | Keltrol ® T | 0.2 | 0.2 | 0.2 | 0.2 | 0.2 | 0.2 |
| | NaOH 1N | — | 0.2 | 0.2 | 0.1 | 0.1 | — |

¹⁾Ryoto L595

To prepare the emulsion, phase 1 was introduced first at 75° C. and phase 2—heated to the same temperature—was added with vigorous stirring. After dilution to 5% by weight, the emulsions had a pH of 6.2 to 7.2 and a Brookfield viscosity of 200 to 600 ps.

TABLE 14
Foaming preparations (figures = % by weight)

| Ingredient | | XC1 | C2 | C3 | C4 | C5 | C6 |
|---|---|---|---|---|---|---|---|
| Phase 1 | Fructose caprate | 2.5 | — | — | — | — | — |
| | Glucose caprylate | | 2.5 | — | — | — | — |
| | Glucose laurate | — | — | 2.5 | — | — | — |
| | Trehalose caprate | — | — | — | 2.5 | — | — |
| | Trehalose laurate | — | — | — | — | 2.5 | — |
| | Sucrose laurate¹⁾ | — | — | — | — | — | 6.25 |
| | Elestab ® 388 | 2.5 | 2.5 | 2.5 | 2.5 | 2.5 | 2.5 |
| | Water | to 100 | | | | | |
| Phase 2 | Sodium Laureth | 30 | 30 | 30 | 30 | 30 | 30 |
| | Sulfate | | | | | | |
| | Cocamidopropyl | 6 | 6 | 6 | 6 | 6 | 6 |
| | Betaine | | | | | | |
| | Sodium chloride | 0.5 | 0.5 | 0.5 | 0.5 | 0.5 | 0.5 |

¹⁾Sisterna L 70C

To produce the preparations, phase 1 was introduced first at 75° C., the Sodium Laureth Sulfate was stirred in and, after cooling to room temperature, the other constituents of phase II were added. After dilution to 5% by weight, the emulsions had a pH of 6 to 7 and a Brookfield viscosity of 200 to 2500 cps.

1-16. (canceled)

17. A method comprising: (a) providing a composition comprising a sugar ester; and (b) contacting a substrate selected from the group consisting of skin and hair with the composition.

18. The method according to claim 17, wherein the sugar ester comprises a fatty acid ester of a sugar selected from the group consisting of monosaccharides, disaccharides and mixtures thereof.

19. The method according to claim 17, wherein the sugar ester comprises a fatty acid ester of a sugar selected from the group consisting of fructose, glucose, trehalose, sucrose and mixtures thereof.

20. The method according to claim 17, wherein the sugar ester comprises a fatty acid ester of fructose.

21. The method according to claim 17, wherein the sugar ester comprises a fatty acid ester of a sugar, the fatty acid moiety corresponding to the general formula R¹CO—, wherein R¹CO is a linear or branched, saturated or unsaturated acyl or hydroxyacyl group having from 6 to 22 carbon atoms and having up to 3 carbon-carbon double bonds.

22. The method according to claim 21, wherein R¹CO is a linear or branched, saturated or unsaturated acyl or hydroxyacyl group having from 8 to 16 carbon atoms and having up to 3 carbon-carbon double bonds.

23. The method according to claim 17, wherein the sugar ester comprises a fatty acid ester of a sugar, the fatty acid moiety comprising a dicarboxylic acid having from 2 to 22 carbon atoms.

24. The method according to claim 17, wherein the sugar ester comprises a fatty acid ester of a sugar, the fatty acid moiety comprising a dicarboxylic acid having from 6 to 18 carbon atoms.

25. The method according to claim 17, wherein the sugar ester comprises a plurality of sugar esters having an average degree of esterification of from 1 to 6.

26. The method according to claim 17, wherein the sugar ester comprises a plurality of sugar esters having an average degree of esterification of from 1. to 2.5.

27. The method according to claim 17, wherein the sugar ester is present in an amount of from 0.0001 to 10% by weight, based on the composition.

28. The method according to claim 17, wherein the sugar ester is present in an amount of from 0.001 to 5% by weight, based on the composition.

29. The method according to claim 17, wherein the sugar ester is present in an amount of from 0.01 to 1% by weight, based on the composition.

30. A method comprising: (a) providing a composition comprising a plurality of sugar esters having an average degree of esterification of from 1. to 2.5, at least one of the sugar esters comprising a fatty acid ester of a sugar selected from the group consisting of fructose, glucose, trehalose, sucrose and mixtures thereof, the fatty acid moiety corresponding to the general formula R¹CO—, wherein R¹CO is a linear or branched, saturated or unsaturated acyl or hydroxyacyl group having from 6 to 22 carbon atoms and having up to 3 carbon-carbon double bonds, and wherein the plurality of sugar esters is present in an amount of from 0.0001 to 10% by weight, based on the composition; and (b) contacting a substrate selected from the group consisting of skin and hair with the composition.
